(19) United States
(12) Patent Application Publication
(10) Pub. No.: US 2023/0017943 A1
(43) Pub. Date: Jan. 19, 2023

(54) MONITORING THE OPERATION OF RESPIRATORY SYSTEMS

(71) Applicant: INTERSURGICAL AG, Vaduz (LI)

(72) Inventors: Andrew Neil MILLER, Wokingham Berkshire (GB); Mark Richard BROWNE, Wokingham Berkshire (GB); Rhys Patrick BRYANT, Wokingham Berkshire (GB)

(21) Appl. No.: 17/786,884

(22) PCT Filed: Dec. 17, 2020
(86) PCT No.: PCT/EP2020/086729

§ 371 Date: Jun. 17, 2022

(30) Foreign Application Priority Data
Dec. 20, 2019 (GB) 1919072.7

(51) Int. Cl. A61M 16/00; A61M 16/06; G16H 40/40; G16H 20/40

(52) U.S. Cl. CPC A61M 16/024; A61M 16/06; A61M 16/0051; G16H 40/40; G16H 20/40; A61M 2016/003; A61M 2016/0027; A61M 2205/18

(57) ABSTRACT

There is provided a method of detecting a fault in a breathing system. The method comprises the steps of (a) taking a series of measurements of a first parameter of the breathing system; and (b) setting a fault boundary for the first parameter, the fault boundary being dependent on a plurality of the measurements of the first parameter. The method further includes at least one update procedure comprising the steps of (c) taking one or more further measurements of the first parameter; and (d) updating the fault boundary, the updated fault boundary being dependent on an updated set of measurements of the first parameter, the updated set of measurements of the first parameter including at least one of the further measurements of the first parameter.

The present invention relates to monitoring the operation of respiratory systems, and more particularly for, but not limited to, monitoring the operation of respiratory systems having gas supplied by constant flow therapy devices such as gas blenders, to identify when a fault or error occurs.

Air/Oxygen blenders are known for use in a variety of healthcare applications to offer a reliable and accurate method of delivering the gas to the patient. In hospitals, for example, gases are generally provided from a supply at the wall, and these gases need to be mixed in desired proportions and delivered to a patient at a specific pressure and/or flow rate.

The ability to provide mixed air and oxygen in a safe, easy and controlled manner is becoming increasingly important. It is therefore critical to know if/when there is a fault in delivery of gas from the device to a patient.

Certain faults with the system have a large effect on a measurable parameter. This allows fault boundaries, ie the boundaries for the measurable parameter that when crossed indicate there is a fault with the system associated with the device, to be set further from a normal value associated with the measurable parameter and still detect faults. However, other faults with the system have less of an effect on the measurable parameter, meaning that fault boundaries would have to be set close to the normal value of the measurable parameter in order to detect faults.

In order to detect such faults, the fault boundaries need to be particularly narrow. However, since the system is used with varying equipment attachments and patient interfaces throughout its lifetime, it is generally not possible to predetermine the required fault boundaries when manufactured, without incurring a large number of false errors. As a result, fault boundaries have to be set particularly wide, with extreme upper and lower fault boundaries, in an attempt to capture all potential system faults, which is often ineffective.

A typical solution to this problem is to allow the operator to set the fault boundaries once the designated equipment has been attached to the device for operation. This allows the fault boundaries to be set more narrowly to detect the aforementioned more subtle potential faults. However, this places a burden on the operator to update the fault boundaries and requires the operator to both a) be aware of the correct boundaries for each piece of attachable equipment, and b) implement those boundaries correctly.

There has now been devised an improved method for monitoring operation of devices, which overcomes or substantially mitigates the abovementioned and/or other disadvantages associated with the prior art.

According to a first aspect of the invention there is provided a method of detecting a fault in a breathing system, the method comprising the steps of

(a) taking a series of measurements of a first parameter of the breathing system; and

(b) setting a fault boundary for the first parameter, the fault boundary being dependent on a plurality of the measurements of the first parameter;

wherein the method includes at least one update procedure comprising the steps of:

(c) taking one or more further measurements of the first parameter; and

(d) updating the fault boundary, the updated fault boundary being dependent on an updated set of measurements of the first parameter, the updated set of measurements of the first parameter including at least one of the further measurements of the first parameter.

This method is advantageous in that it allows the accurate setting of a fault boundary regardless of the configuration of the breathing system. The fault boundary for the first parameter being dependent on measurements of the first parameter taken in the previous step of the method ensures that the fault boundary is appropriate regardless of the equipment or patient interface being used with the breathing system.

Furthermore, updating the fault boundary dependent on further measurements of the first parameter taken ensures that the fault boundary continues to remain appropriate throughout use of the system. This is particularly advantageous, for example, since the breathing rate of a patient and any movement of the components of the breathing system are not constant, varying particularly based on whether the patient is asleep or in a period of wakefulness. Thus, updating the fault boundary allows the state of the patient and/or any movement of the components of the breathing system to be taken into account when judging what should and shouldn't be considered a fault.

Setting of the fault boundary for the first parameter may comprise setting of the fault boundary to be offset from a measurement parameter that is dependent on a plurality of the measurements. After taking one or more further measurements, the measurement parameter may be updated. The updated measurement parameter may be dependent on at least one of the further measurements.

The measurement parameter may be an average of a plurality of the measurements. Alternatively, the measurement parameter may be a minimum and/or maximum value of the series of measurements. After taking the series of measurements of the first parameter, the method may further comprise a step of determining an average of a plurality of the measurements. Determining an average of a plurality of the measurements may comprise calculating a mean value or a median value of a plurality of the measurements, or any other functionally equivalent calculation of a central value. Where the measurement parameter has been updated, the updated measurement parameter may be an average of a plurality of the updated set of measurements or a minimum and/or maximum value of the updated set of measurements.

Setting of the fault boundary or the updated fault boundary for the first parameter may comprise setting of the fault boundary relative to the measurement parameter or the updated measurement parameter, eg the determined average, the minimum and/or maximum value of the series of measurements, the average of a plurality of the updated set of measurements or the minimum and/or maximum value of the updated set of measurements. Setting of the fault boundary may comprise setting an upper fault boundary and/or a lower fault boundary. The upper fault boundary may be greater than the measurement parameter, or where the upper fault boundary is an updated upper fault boundary, the updated measurement parameter and the lower fault boundary may be less than the measurement parameter, or where the lower fault boundary is an updated lower fault boundary, the updated measurement parameter. For example, the upper fault boundary may be greater than the determined average or updated average and the lower fault boundary may be less than the determined average or updated average.

The fault boundary may be offset from the measurement parameter by a predetermined amount. Alternatively, the fault boundary may be offset from the measurement parameter by an amount that is dependent on a variance factor of a plurality of the measurements. The fault boundary may be offset from the measurement by a fraction or a percentage of the determined average. Where the fault boundary is an updated fault boundary, the offset of the updated fault boundary from the updated measurement parameter may be a predetermined amount or an amount that is dependent on an updated variance factor of a plurality of the updated set of measurements.

After taking the series of measurements of the first parameter, the method may further comprise a step of determining a variance factor of a plurality of the measurements. The variance factor may be dependent on the variance of a plurality of the measurements. The variance factor may be dependent on the variance of a plurality of the measurements about a measurement parameter, for example the determined average.

The variance factor may comprise the difference between the highest and lowest measurements of the plurality of measurements. Alternatively, the variance factor may comprise the greatest difference between any one of the plurality of measurements and the measurement parameter, for example the determined average. Alternatively, the variance factor may comprise an upper variance factor and a lower variance factor, the upper variance factor being the greatest difference between the measurement parameter and any one of the plurality of measurements above the measurement parameter, and the lower variance factor being the greatest difference between the measurement parameter and any one of the plurality of measurements below the measurement parameter. Alternatively, the variance factor may comprise a statistical variance of the plurality of measurements, for example a statistical deviation.

The fault boundary may be offset from the measurement parameter by the variance factor multiplied by an error factor. The error factor may equate to an amount of variance that is acceptable during normal operation of the system. Where the determined variance factor comprises an upper variance factor and a lower variance factor, setting of the fault boundary may comprise setting an upper fault boundary that is offset from the measurement by the upper variance factor multiplied by an upper error factor and setting a lower fault boundary that is offset from the measurement parameter by the lower variance factor multiplied by a lower error factor. The upper and lower error factors may be the same or different. Where the fault boundary is an updated fault boundary, the updated fault boundary may be offset from the updated measurement parameter by the updated variance factor multiplied by an error factor.

The updated set of measurements may comprise at least one of the measurements upon which the fault boundary being updated is dependent and at least one of the further measurements. The updated set of measurements may comprise a plurality of the measurements upon which the fault boundary being updated is dependent and at least one of the further measurements. The plurality of the measurements upon which the fault boundary being updated is dependent, in the updated set of measurements, may be the most recently taken measurements of the measurements upon which the fault boundary being updated is dependent. The updated set of measurements may comprise the same number of measurements as the plurality of measurements upon which the fault boundary being updated is dependent. The updated set of measurements may comprise at least one of the further measurements, which replace an equivalent number of the earliest measurements upon which the fault boundary being updated is dependent.

The one or more further measurements of the first parameter may comprise a single further measurement. In this case, the updated set of measurements may comprise all but one of the plurality of the measurements upon which the fault boundary being updated is dependent. The updated set of measurements may comprise all but the earliest of the plurality of the measurements upon which the fault boundary being updated is dependent. Alternatively, the one or more further measurements of the first parameter may comprise a series of further measurements. In this case, in the updated set of measurements, the series of further measurements may replace an equivalent number of the earliest measurements upon which the fault boundary being updated is dependent.

After taking one or more further measurements of the first parameter, the method may further comprise a step of updating the measurement parameter, the updated measurement parameter being dependent on at least one of the further measurements of the first parameter. For example, the method may further comprise a step of updating the determined average, the updated average being dependent on at least one of the further measurements of the first parameter. Updating the determined average may comprise determining an average of the updated set of measurements of the first parameter. This is commonly referred to as maintaining a rolling average of the measurements of the first parameter.

This may be advantageous in that a rolling average of the first parameter is maintained throughout operation of the breathing system, allowing the fault boundary to be updated according to current operation.

After taking one or more further measurements of the first parameter, the method may further comprise a step of updating the determined variance factor, the updated variance factor being dependent on at least one of the further measurements of the first parameter. Updating the determined variance factor may comprise determining a variance factor for the updated set of measurements of the first parameter. The updated variance factor may be dependent on the variance of the updated set of measurements. The updated variance factor may be dependent on the variance of the updated set of measurements about the updated measurement parameter, for example the updated average.

The updated variance factor may comprise the difference between the highest and lowest measurements of the updated set of measurements. Alternatively, the updated variance factor may comprise the greatest difference between any one of the updated set of measurements and the updated measurement parameter, for example the updated average. Alternatively, the updated variance factor may comprise an updated upper variance factor and an updated lower variance factor, the updated upper variance factor being the greatest difference between the updated measurement parameter and any one of the updated set of measurements above the updated measurement parameter, and the updated lower variance factor being the greatest difference between the updated measurement parameter and any one of the update set of measurements below the determined measurement parameter. Alternatively, the updated variance factor may comprise a statistical variance of the updated set of measurements, for example a statistical deviation.

Updating the determined variance factor dependent on further measurements of the first parameter taken ensures that the fault boundary continues to remain appropriate throughout use of the system. This is particularly advantageous when considering the varying breathing rate of a patient and the varying movement of the components of the breathing system, which are highly dependent on whether the patient is asleep or in a period of wakefulness. When the patient is asleep for example, their breathing rate does not vary much, and the breathing system remains relatively still, thus a lower variance factor is determined, and narrower boundaries can be set so that smaller changes that could indicate a blockage or leak can be detected. On the other hand, when the patient is in a period of wakefulness, or is generally more restless, their breathing rate varies more, and the breathing system moves more, hence a higher variance factor is determined and wider boundaries are set so that false alarms are less likely to occur.

Thus, updating the fault boundary allows the state of the patient to be taken into account when judging what should and shouldn't be considered a fault.

Updating the fault boundary for the first parameter may comprise updating the fault boundary relative to the updated measurement parameter. For example, updating the fault boundary for the first parameter may comprise updating the fault boundary relative to the updated average. Updating the fault boundary may comprise updating the fault boundary dependent on the updated variance factor. Updating the fault boundary may comprise updating an upper fault boundary and updating a lower fault boundary.

The method may further comprise comparing a measurement of the first parameter with the fault boundary, or where the fault boundary has been updated, with the updated fault boundary, and determining a fault with the operation of the breathing system in response to the measurement being outside of the fault boundary or the updated fault boundary. Where the fault boundary comprises an upper fault boundary and a lower fault boundary, the method may comprise comparing a measurement of the first parameter with the upper fault boundary, or where the upper fault boundary has been updated, with the updated upper fault boundary and determining a fault with the operation of the breathing system in response to the measurement being above the upper fault boundary or the updated upper fault boundary, and comparing a measurement of the first parameter with the lower fault boundary, or where the lower fault boundary has been updated, with the updated lower fault boundary, and determining a fault with the operation of the breathing system in response to the measurement being below the lower fault boundary or the updated lower fault boundary. Where the fault boundary has been updated, comparison of a measurement of the first parameter may be with the latest updated fault boundary.

In response to determining a fault with the operation of the breathing system, the method may further comprise the step of indicating the fault. Additionally or alternatively, the breathing system may act to adjust its parameters and/or method of operation and/or state of operation accordingly.

The update procedure may be repeated at least once. The fault boundary being updated in each subsequent update procedure may be the updated fault boundary from the previous update procedure. The update procedure may be repeated at intervals throughout operation of the breathing system. Operation of the breathing system may be considered the period during which gas is supplied to the breathing system, and/or the period during which treatment is being provided to a patient. The update procedure may be repeated at intervals following an initial calibration process for any of, or any combination of the breathing system, a device supplying gas to the breathing system, and a device for carrying out the method according to the first aspect of the invention.

The update procedure may be repeated until any of, or any combination of: turning off of the breathing system or device, changing of any attached equipment that requires a resetting of the fault boundary, changing of any parameter settings, a change in the treatment carried out by the breathing system or device, and user acknowledgement of an existing fault.

The intervals may be regular intervals. The update procedure may be repeated at least three times, at least five times, at least ten times, at least 50 times, or at least 100 times. The update procedure may be repeated every 0.01 seconds, every 0.1 second, every second, every two seconds, every five seconds, every ten seconds, or every fifteen seconds. The intervals may be less than 0.01 seconds, less than 0.1 second, less than one second, less than two seconds, less than five seconds, less than ten seconds, or less than fifteen seconds.

Step (a) and/or step (b) of the first aspect of the invention may occur during operation of the breathing system, ie the period during which gas is supplied to the breathing system, and/or the period during which treatment is being provided to a patient. Before step a) of the first aspect of the invention, the method may comprise the step of commencing supplying gas to the breathing system. The method may further comprise the step of continuing supplying gas to the breathing system whilst conducting steps (a)-(d) of the first aspect of the invention. Step (a) may commence almost immediately after commencing supplying gas to the breathing system.

The term “almost immediately” may account for a slight delay between commencing supplying gas to the breathing system and starting step (a) of the first aspect of the invention, which allows the supplied gas to move through most or all of the breathing system before the series of measurements is taken. In use, the breathing system may be connected to an apparatus for supplying gas, which may comprise a gas bottle or cannister, or a fixed gas line in a hospital wall, for example. Supply of the gas from the gas-supply apparatus to the breathing system may be controlled by a suitable device such as a gas blender. Supply of the gas from the gas-supply apparatus to the breathing system may be controlled by at least one valve for controlling the gas supply. The suitable device, eg a gas blender, may comprise the at least one valve. Commencing supplying gas to the breathing system may therefore comprise opening of the at least one valve. For example, the apparatus for supplying gases may already be turned “on”, ie the apparatus may be arranged such that it can readily supply gas, but the gas is not supplied to the breathing system until the valve is opened.

According to a further aspect of the invention there is provided a device configured to implement the method according to the first aspect of the invention. The device may be a device for supplying gas to the breathing system. The gas may be respiratory gas.

The device for supplying gas may comprise an inlet, at which gas is provided to the device, and an outlet, from which gas is supplied to the breathing system to which the device is connected. The breathing system may comprise a patient delivery tube and a patient interface, the delivery tube arranged to supply gas from the device to the patient interface. The patient interface may comprise a connector configured to connect to the patient delivery tube. The patient delivery tube may have a first end for connection to the outlet of the device, and a second end for connection to the patient interface. The patient interface may be arranged to deliver gas supplied from the device to a patient, via the patient delivery tube. The patient interface may be a respiratory mask, for example.

The first parameter may be dependent on faults in the configuration of the breathing system, such as a kink in the patient delivery tube, blockage of the patient delivery tube, disconnection or removal of a component of the breathing system, or a leak in the breathing system. This may be advantageous in that it enables detection of these occurrences to be recognised as a fault with the operation of the breathing system.

The method may be further advantageous in that it enables these faults with operation of the breathing system to be recognised regardless of the type of breathing system connected to the device for supplying gas.

The first parameter may be a parameter of the gas supply within the breathing system. The first parameter may be a parameter of the breathing system, which may be measured at the device. The first parameter may be dependent on the configuration of the breathing system. A change in the first parameter may therefore be indicative of a change in the configuration of the breathing system. A change in the first parameter may be indicative of a fault in the breathing system. The first parameter may be a parameter associated with the effect the breathing system has on the gas supply within the device. The first parameter may be a parameter that is not controlled at the device. That is, the first parameter may be unregulated and uncalibrated by the device. The first parameter may be a pressure within the device or the breathing system. The first parameter may be measured at the device, for example within the device, at an outlet of the device, or at an inlet of the device. Alternatively, the first parameter may be measured within the breathing system, for example within the breathing tube, or at the patient interface.

The first parameter may be any of flow rate, back pressure within the breathing system, or patient pressure.

The back pressure may be defined as the difference between the pressure at any point within the breathing system and the ambient pressure, ie the environmental pressure surrounding the breathing system. The patient interface of the breathing system may be exposed to ambient pressure. In use, the pressure at any point within the breathing system should be higher than ambient pressure to ensure the flow of gas through the breathing system to supply gas from the device to the patient interface. The ambient pressure may be atmospheric pressure. It may be assumed that the atmospheric pressure is fixed, for example at approximately one atmosphere, approximately one bar, or approximately 100 kPa. Where the atmospheric pressure is taken to be fixed, a pressure within the device may be measured rather than the back pressure within the breathing system. The back pressure may arise as a result of gas flowing through a resistance in the breathing system. That is, the back pressure may be dependent on, and therefore vary as a function of, the resistance to flow of gas through the breathing system. An increase in the resistance to flow may therefore increase the back pressure within the breathing system. The back pressure may also be dependent on, and therefore vary as a function of, flow rate. Similarly, the patient pressure may be defined as the pressure in the proximity of a patient interface of the breathing system, for example at the patient interface.

The method may further comprise controlling a second parameter of the breathing system. Control of the second parameter of the breathing system may involve control of the device for supplying gas to the breathing system. The first parameter may additionally be dependent on the second parameter.

The control of a second parameter of the breathing system and the first parameter being dependent on the second parameter may provide further advantages over the prior art. Hence, according to a further aspect of the invention, there is provided a method of detecting a fault in a breathing system, the method comprising the steps of:

(a) supplying a gas to the breathing system;

(b) taking a series of measurements of a first parameter of the breathing system;

(c) setting a fault boundary for the first parameter, the fault boundary being dependent on a plurality of the measurements of the first parameter,

wherein a second parameter of the breathing system is controlled during supply of the gas to the breathing system, and the first parameter of the breathing system is dependent on the second parameter and the breathing system, such that a change in the first parameter may be indicative of a fault in the breathing system.

Controlling the second parameter of the gas supply at the device may comprise supplying the device with gas having a predetermined value for the second parameter. Controlling the second parameter of the gas supply at the device may comprise measuring a value of the second parameter at a first position in the device, for example at an outlet of the device. Controlling the second parameter of the gas supply at the device may comprise regulating and/or calibrating the value of the second parameter of the gas supply at a second position of the device in response to the measured value of the second parameter at the first position. It will be appreciated that the positions above are referred to as a first position and a second position to distinguish between those measurements, not necessarily to indicate their position in the device relative to one another.

The second parameter may be any parameter that may be controlled at the device. The second parameter may be any parameter of the gas supply that may be set or defined at the device, or when supplied to the device. The second parameter may be measurable at the device, for example at the inlet or the outlet of the device. The second parameter may be measurable in the breathing system, for example in the patient delivery tube, or at the patient interface. The second parameter may be measurable by a sensor. The second parameter may be regulated and/or calibrated by the device. The second parameter may be flow rate, since this is typically the parameter controlled by the device for supplying gas. However, it may also be possible for the second parameter to be a pressure within the device or the breathing system, for example pressure at an outlet of the device, back pressure within the breathing system, or patient pressure.

The device may have a second fault detection system for monitoring the operation of the breathing system. The second fault detection system may signal a fault in response to changes in the second parameter. For example, where the difference between a measurement of the second parameter, or an average of a plurality of measurements of the second parameter, and a pre-determined target value for the second parameter, exceeds a fault threshold. The method according to the present invention may therefore be implemented in conjunction with another fault detection system of the device itself.

In use, where the second parameter is flow rate and the first parameter is back pressure within the breathing system, or a pressure that is indicative of the back pressure within the breathing system, and there is a change in the breathing system, eg a kink in the patient delivery tube, which reduces the flow rate momentarily, the device will typically provide increased power to the conveying of gases into the breathing system in order to maintain a constant flow rate. In this arrangement, the change in the breathing system, eg the kink in the patient delivery tube, will have caused the back pressure within the breathing system to increase. The method according to the present invention will therefore signal a fault in the event that the back pressure within the system falls outside the at least one boundary, whereas the second fault detection system would not signal a fault, since the flow rate is maintained successfully.

On the other hand, if the device cannot provide a sufficiently increased power to the conveying of gases into the breathing system in order to maintain a constant flow rate, the method according to the present invention may not signal a fault, since there may not be a sufficient increase in back pressure for the method according to the present invention to signal a fault, whereas the second fault detection system of the device would signal a fault as a result of the reduced flow rate. The increased power provided by the device may equate to an increased supply pressure.

The first parameter may be dependent on, and therefore reflective or indicative of, a third parameter. A change in the first parameter may be indicative of a change in the third parameter. The third parameter may be dependent on the configuration of the breathing system. For example, the third parameter may be dependent on the fault conditions referred to above. The third parameter may be back pressure within the breathing system, or patient pressure, for example. This may be advantageous in that it allows a simple measurement of the first parameter to be taken within the device or the breathing system, any variation in the first parameter being indicative of a variation in the third parameter, the third parameter typically being more difficult to measure, but being indicative of a fault within the breathing system.

The first parameter may be a pressure within the device. The third parameter may be back pressure within the breathing system. The first parameter may be determined by measuring the pressure at a position within the device, since when the ambient pressure is fixed, the pressure offset from the ambient pressure will be indicative of the back pressure through the breathing system, allowing the back pressure within the breathing system to be determined. There may therefore be no requirement for further processing or calculation to obtain the back pressure.

The series of measurements may be measured over a first period of time. The first period of time may be at least ten seconds, at least twenty seconds, at least thirty seconds, at least forty-five seconds, at least one minute, at least two minutes, at least three minutes, at least four minutes, or at least five minutes. Measuring the first parameter for the first period of time may comprise measuring the parameter at least every 0.01 seconds, at least every 0.1 second, at least every second, at least every two seconds, at least every five seconds, at least every ten seconds, or at least every fifteen seconds.

Measuring the first parameter may comprise measuring the first parameter using at least one sensor. The at least one sensor may comprise a plurality of sensors positioned at different positions throughout the device and each measurement of the first parameter may comprise taking an instantaneous average of the measurements measured by the plurality of sensors.

The first parameter may be dependent on any, or any combination of, the number or type of components of the breathing system, the type of device for supplying gas, the mode of operation of the device and/or the breathing system, and the treatment type provided by the breathing system. The first parameter may also be dependent on the fault conditions referred to above.

Where the device utilises a plurality of sensors, determining an average of a plurality of the measurements may comprise averaging the instantaneous averages measured over the first period of time, ie calculating the mean or median value of the instantaneous averages measured over the first period of time. In exemplary embodiments, the fault boundary may be offset from the determined average by between 5 and 50%, between 10% and 30%, or between 15 and 25%, for example by 5%, by 10%, by 15%, by 20%, by 25%, or by 30%. In further exemplary embodiments, the fault boundary may be offset from the determined average by one times the determined variance factor, two times the determined variance factor, three times the determined variance factor, or four times the determined variance factor.

The offset of the fault boundary from the measurement parameter, or where the fault boundary has been updated, the offset of the updated fault boundary from the updated measurement parameter, may be compared with a predetermined minimum offset. The predetermined minimum offset may be associated with the at least one sensor measuring the first parameter. The predetermined minimum offset may be associated with the measuring accuracy of the at least one sensor measuring the first parameter. That is, the predetermined minimum offset may be a selected value associated with the measuring accuracy of the at least one sensor measuring the first parameter. The selected value may be the minimum difference required between any two measurements for the at least one sensor measuring the first parameter to distinguish the two values without error, based on its measuring accuracy. Where the offset of the fault boundary or the offset of the updated fault boundary is less than the predetermined minimum offset, the offset of the fault boundary or the updated fault boundary may be increased to match the predetermined minimum offset.

This may be advantageous in that it ensures the boundaries are wide enough to prevent a large number of false errors from being signalled.

The offset of the lower fault boundary and/or the upper fault boundary from the determined average, or where the lower fault boundary and upper fault boundary have been updated, the offset of the updated lower fault boundary and/or the updated upper fault boundary from the updated average, may be compared with a maximum offset. Where the offset of the lower fault boundary and/or the upper fault boundary, or where the lower fault boundary and upper fault boundary have been updated, the offset of the updated lower fault boundary and/or the updated upper fault boundary, is more than the maximum offset, the offset of the lower fault boundary and/or the upper fault boundary, or where the lower fault boundary and upper fault boundary have been updated, the offset of the updated lower fault boundary and/or the updated upper fault boundary, may be decreased to match the maximum offset.

The upper fault boundary may be a first upper fault boundary and the lower fault boundary may be a first lower fault boundary. Second upper and lower fault boundaries may also be implemented. The second upper and lower fault boundaries may be conventional wider fault boundaries. The second upper fault boundary may be greater than the first upper fault boundary, and the second lower fault boundary may be less than the first lower fault boundary. Alternatively, the second upper fault boundary may be equal to or less than the first upper fault boundary, and the second lower fault boundary may be equal to or more than the first lower fault boundary. This is possible as a result of the dynamic nature of the first upper fault boundary and the first lower fault boundary, and their dependence on the further measurements of the first parameter.

Comparing a measurement of the first parameter with the fault boundary or updated fault boundary may comprise continuing to take further measurements of the first parameter and comparing each measurement with the fault boundary. Further measurements may be taken every 0.01 seconds, every 0.1 second, every second, every two seconds, every five seconds, every ten seconds, or every fifteen seconds. Signalling a fault with the operation of the device may comprise raising an alarm signal. The alarm signal may comprise any or any combination of an audio signal, a visual signal, and a vibration.

Where second upper and lower fault boundaries are implemented, a different fault with the operation of the device may be determined in response to the first parameter exceeding the second upper fault boundary or being lower than the second lower fault boundary. In this example, a different signal may be raised in response to the first parameter exceeding the second upper fault boundary or being lower than the second lower fault boundary.

The method may update the determined average of the first parameter only where the one or more further measurements of the first parameter falls inside of the fault boundary. Where the average is an updated average, the method may update the updated average only where the one or more further measurements of the first parameter falls inside of the updated fault boundary from the previous update procedure.

The method may update the determined average after each of the one or more further measurements of the first parameter, or after a series of further measurements of the first parameter. For example, if the first parameter is measured every 0.1 seconds, the average of the first parameter and the variance factor of the measurements about the determined average of the first parameter may be updated every 10 measurements, ie every second.

This is advantageous in that the at least one boundary associated with the first parameter is dynamically updated in respect of the most recent measurements of the first parameter.

In response to determining a fault with the operation of the breathing system, the device may pause or shut down such that operation of the device ceases. The method of monitoring the operation of the breathing system may also cease. The method of monitoring the operation of the breathing system may restart with the step of setting a second parameter once the fault with operation has been solved. The fault with operation of the breathing system may be solved automatically by the device or by an operator. Alternatively, the breathing system may continue in operation and the method of monitoring the operation of the breathing system may also continue. Where this is the case, the alarm signal may continue to be raised until there is no longer a fault with the operation of the breathing system.

The method of monitoring the operation of the breathing system may also restart with the step of setting a second parameter in response to any of, or any combination of: turning off of the breathing system or device, changing of any attached equipment that requires a resetting of the fault boundary, changing of any parameter settings, a change in the treatment carried out by the breathing system or device, or user acknowledgement of an existing fault.

The device may be a gas blender. The gas blender may form part of an anaesthetic machine or a stand-alone unit arranged to be connected to a gas supply. The gas blender may comprise input ports for gas supplies from pressure lines. Alternatively, the device may be a continuous positive airway pressure (CPAP) driver, or an infant flow driver.

According to a further aspect of the invention there is provided a computer-readable storage medium comprising instructions which, when executed by a data processor, cause the data processor to carry out the method as defined above.

According to a further aspect of the invention there is provided a respiratory apparatus comprising a controller and at least one sensor configured to carry out the method as defined above.

The respiratory apparatus may further comprise at least one indicator configured to signal a fault with the operation of the breathing system in response to a further measurement being outside of the fault boundary.

The respiratory apparatus may be integrated with the breathing system being monitored. The apparatus may be integral with the device supplying gas to the breathing system. Alternatively, the respiratory apparatus may be remote from the device and the breathing system being monitored.

According to a further aspect of the invention there is provided a respiratory apparatus for supplying gas to a breathing system, the respiratory apparatus comprising: at least one sensor configured to take a series of measurements of a first parameter of the breathing system and configured, in at least one update procedure, to take one or more further measurements of the first parameter; a controller configured to set a fault boundary for the first parameter, the fault boundary being dependent on a plurality of the measurements of the first parameter, and configured, in the at least one update procedure, to update the fault boundary, the updated fault boundary dependent on an updated set of measurements of the first parameter, the updated set of measurements of the first parameter including at least one of the further measurements of the first parameter.

The controller may comprise a processor. The controller may be configured to compare a measurement of the first parameter with the fault boundary. The controller may be further configured to signal a fault with the operation of the breathing system in response to the measurement being outside of the fault boundary. The respiratory apparatus may further comprise at least one indicator configured to signal a fault with the operation of the breathing system in response to the measurement being outside of the at least one boundary.

The controller may be further configured to determine an average of the plurality of the measurements. The controller may be further configured to determine a variance factor of the plurality of the measurements

The controller may be configured to control a second parameter of the gas supply at the respiratory apparatus. The first parameter may additionally be dependent on the second parameter. Controlling the second parameter of the gas supply may comprise supplying the device with gas having a predetermined or user-selectable value for the second parameter.

The respiratory apparatus may further comprise at least one sensor configured to measure the second parameter. This at least one sensor may communicate with the controller of the respiratory apparatus so as to regulate and/or calibrate the second parameter. This communication may be via the controller.

The respiratory apparatus may further comprise a user interface configured to indicate to a user any or any combination of the set second parameter value, the average of the plurality of measurements of the first parameter, the determined variance factor of the plurality of measurements of the first parameter, the last measured first parameter value and the fault boundary.

The at least one indicator may comprise a speaker for providing an audio signal in response to the first parameter falling outside of the fault boundary. The at least one indicator may comprise a light source for providing a visual signal in response to the first parameter falling outside of the fault boundary. Alternatively, a visual signal may be provided on the user interface. The at least one indicator may comprise a plurality of indicators, ie a combination of the aforementioned indicators.

The respiratory apparatus may further comprise an inlet. The inlet may be arranged to receive gas from an external gas supply. The inlet may be configured to connect or attach to an external gas supply. The external gas supply may be in the form of a gas bottle or cannister, or a fixed gas line in a hospital wall, for example. The respiratory apparatus may further comprise an outlet. The outlet may be arranged to supply gas to the breathing system. The outlet may be configured to connect or attach to the breathing system, such that the respiratory apparatus is in fluid communication with the breathing system. The breathing system may comprise a patient interface for providing the gas supply to a patient. The breathing system may comprise a breathing tube arranged to deliver the gas supply from the outlet to the patient interface.

According to a further aspect of the present invention, there is provided a respiratory system comprising the respiratory apparatus as defined above, and a breathing system in fluid communication with the respiratory apparatus, the breathing system comprising a patient interface arranged to supply gas to a patient in use, and a breathing tube arranged to deliver the gas supply from the respiratory apparatus to the patient interface.

According to a further aspect of the invention, there is provided a respiratory system comprising a breathing system, and a respiratory apparatus for supplying gas to the breathing system, the respiratory apparatus comprising: at least one sensor configured to take a series of measurements of a first parameter of the breathing system and configured, in at least one update procedure, to take one or more further measurements of the first parameter; a controller configured to set a fault boundary for the first parameter, the fault boundary being dependent on a plurality of the measurements of the first parameter, and configured, in the at least one update procedure, to update the fault boundary, the updated fault boundary dependent on an updated set of measurements of the first parameter, the updated set of measurements of the first parameter including at least one of the further measurements of the first parameter; the breathing system comprising: a patient interface arranged to supply gas to a patient in use, and a breathing tube arranged to deliver the gas supply from the respiratory apparatus to the patient interface.

The controller may comprise a processor. The controller may be further configured to compare a measurement of the first parameter with the fault boundary. The controller may be further configured to signal a fault with the operation of the breathing system in response to the measurement being outside of the fault boundary. The respiratory apparatus may further comprise at least one indicator configured to signal a fault with the operation of the breathing system in response to the measurement being outside of the at least one boundary.

The controller may be further configured to determine an average of the plurality of the measurements. The controller may be further configured to determine a variance factor of the plurality of the measurements

The controller may be further configured to control a second parameter of the gas supply at the respiratory apparatus. The first parameter may additionally be dependent on the second parameter.

The breathing system may further comprise a gas supply connected to the respiratory apparatus such that the gas supply is in fluid communication with the respiratory apparatus. The gas supply may be in the form of a gas bottle or cannister, or a fixed gas line in a hospital wall, for example.

The patient interface may be a face mask, or a nasal mask, for example.

Practicable embodiments of the invention will now be described, by way of example only, with reference to the accompanying drawings, of which:

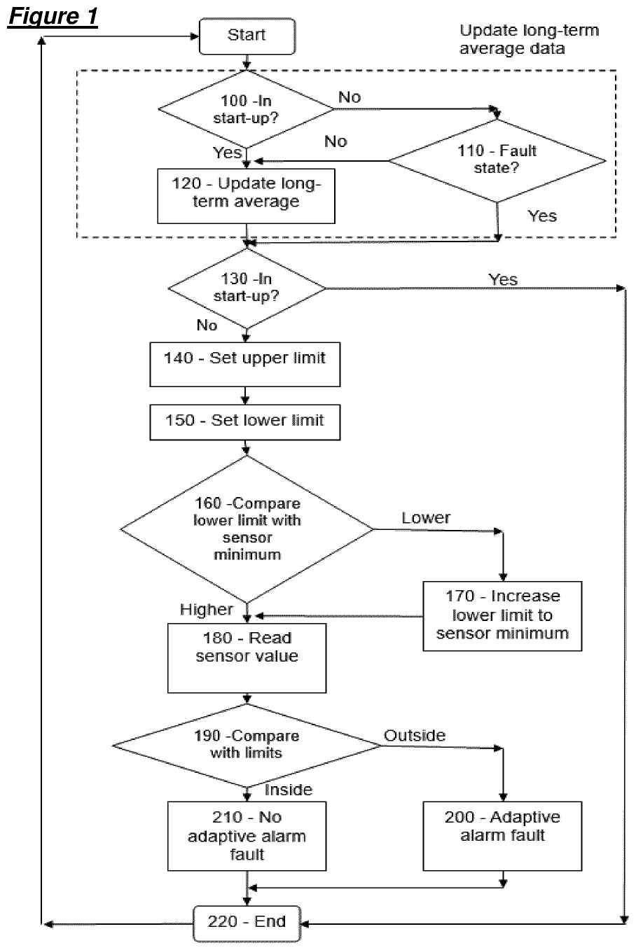

FIG. 1 is a flow diagram illustrating a method of dynamically updating the fault boundaries for a gas parameter in a constant flow therapy device and testing for a fault according to a first embodiment;

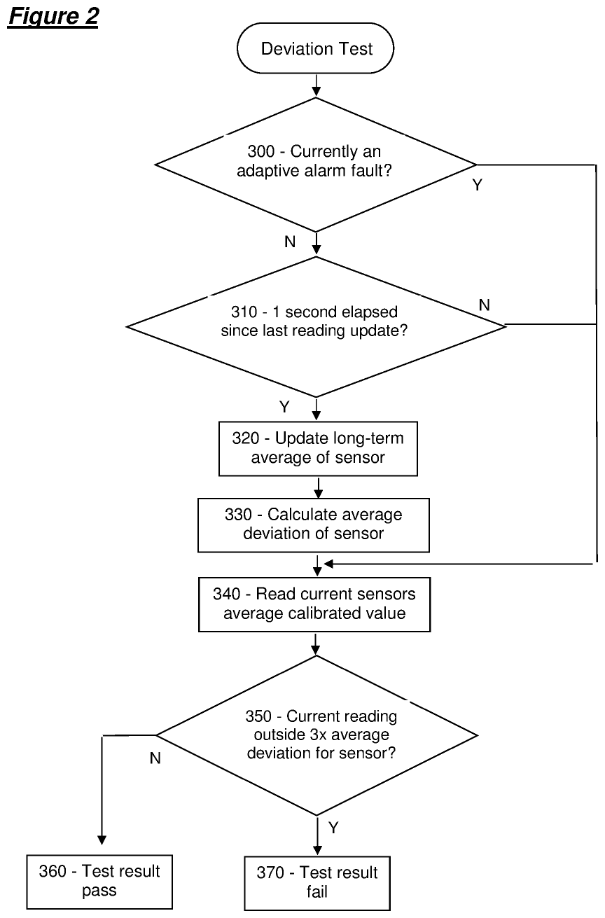

FIG. 2 is a flow diagram illustrating a method of dynamically updating the fault boundaries for a gas parameter in a constant flow therapy device and testing for a fault according to a second embodiment;

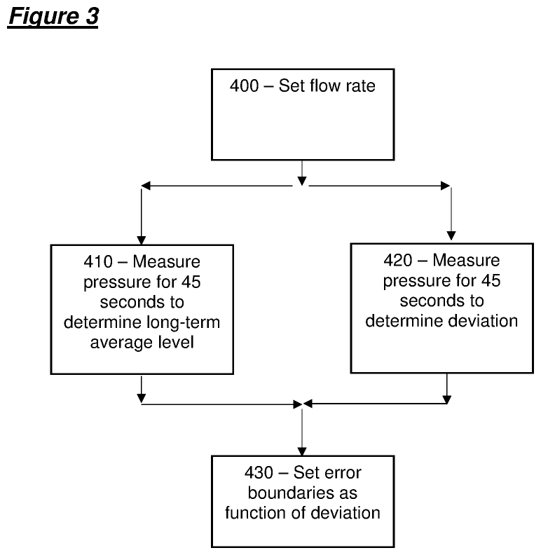

FIG. 3 is a flow diagram illustrating initialisation of the parameters associated with the method of FIG. 2.

FIG. 4 is a first worked example of the setting of fault boundaries for a gas parameter in a constant flow therapy device;

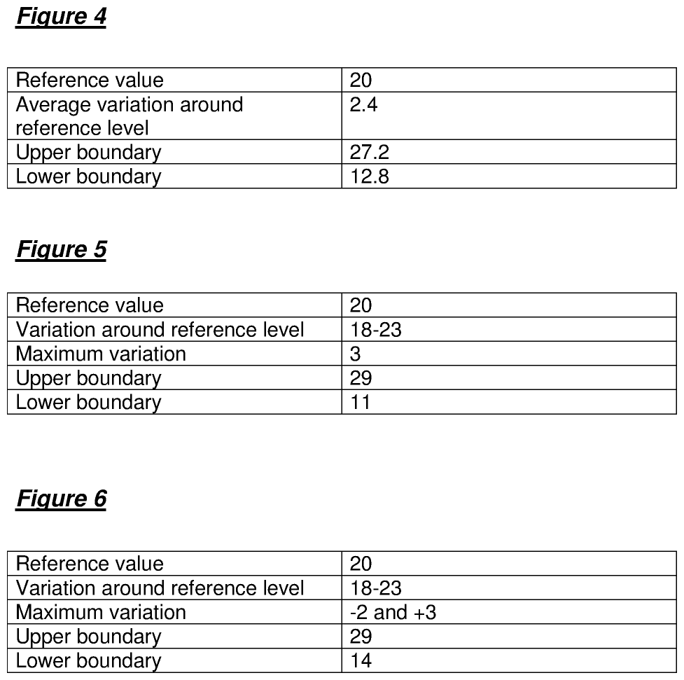

FIG. 5 is a second worked example of the setting of fault boundaries for a gas parameter in a constant flow therapy device;

FIG. 6 is a third worked example of the setting of fault boundaries for a gas parameter in a constant flow therapy device; and

FIG. 1 illustrates a method by which the fault boundaries for a gas parameter in a constant flow therapy device are dynamically updated during operation of the device according to a first embodiment, in order to determine whether there is an error in the operation of the device and then testing for a fault.

Steps 100-120, indicated by the dashed box of FIG. 1, illustrate an initialisation process by which the long-term average of the sensor is determined.

At step 100 it is determined whether the device is in a start-up mode. The start-up mode is defined as being when the device has just been turned on, when the treatment provided by the device has just been changed, or when the parameters of the device have just been altered by an operator. When the device is in the start-up mode, the initialisation process indicated by the dashed box runs as a continuous loop, for 45 seconds. It will be appreciated that the exact value of this time period is not essential, and may vary, within reason.

If at step 100 it is determined that the device is in the start-up mode, the method progresses to step 120, where a measurement of a parameter is taken and the long-term average of that parameter (ie over the 45 second period) is updated.

The measured parameter is one that varies as a result of another parameter that is predefined by a user, ie as a result of using a different patient interface or as a result of variations in the therapy delivered. The measured parameter is also one that varies as a result of faults in a breathing system to which the device is connected. In the following embodiments, this measured parameter will be exemplified as pressure at the device. The pressure at the device is known to be dependent on the back pressure within the breathing system to which the device is connected, and it is known that the back pressure within the breathing system is dependent on any faults in that breathing system. Hence, a change in the pressure at the device indicates a change in the back pressure within the breathing system, which if large enough, may indicate a fault in the breathing system.

Once the long-term average has been updated, the method progresses to step 130. At step 130, it is determined whether the device is still in the start-up mode. If yes, the method returns to the start to continue with the initialisation process.

Once the 45 second initialisation process has been undertaken, at step 130 it will be determined that the device is no longer in the start-up mode, and the method will progress to steps 140 and 150 simultaneously. At steps 140 and 150, an upper fault boundary and a lower fault boundary are defined. These are defined as being 20% higher than the long-term average pressure and 20% lower than the long-term average pressure respectively. It will be appreciated that the exact value of these percentages are not essential, and may vary, within reason.

At step 160, the offset of the lower fault boundary from the long-term average defined in step 150 is compared with a predetermined minimum offset. The predetermined minimum offset is associated with the measuring accuracy of the sensor measuring the pressure at the device. That is, the predetermined minimum offset equates to the minimum difference required between any two measurements for the sensor to distinguish the two values for certain, based on its measuring accuracy. If the lower fault boundary offset is lower than the predetermined minimum offset assigned to the sensor, then at step 170, this is adjusted. This ensures that the upper fault boundary and the lower boundary do not become too narrow about the long-term average, which could lead to a large number of measurements being falsely determined as error measurements, particularly where the precision or accuracy of the sensor is relatively low.

Where it is determined that the offset of the lower fault boundary from the long-term average is lower than the predetermined minimum offset, both the offset of the lower fault boundary and the higher fault boundary are increased to the predetermined minimum offset. As an example, if the long-term average at step 120 is 3, then the 20% fault boundaries will result in an upper boundary of 3.6 and a lower boundary of 2.4. The fault boundary offset of 0.6 will be compared with a predetermined minimum offset assigned to the sensor. If the predetermined minimum offset assigned to the sensor is 2 (ie the sensor is accurate to ±2), then the lower fault boundary offset and the higher fault boundary offset will be increased to 2, such that the lower fault boundary becomes 1 (ie 3−2) and the higher fault boundary becomes 5 (ie 3+2). It will be appreciated that in other examples, only one of the fault boundaries may be offset in response to the offset of the lower fault boundary from the long-term average being lower than the predetermined minimum offset

If the lower fault boundary offset is higher than the predetermined minimum offset assigned the sensor, then the method continues and progresses to step 180.

A maximum offset may also be defined in a similar manner such that the fault boundaries do not become too wide about the long-term average.

At step 180, the sensor takes a pressure measurement. This measurement is compared with the defined fault boundaries at step 190. If the measured pressure is found to be outside of those fault boundaries, then an adaptive alarm fault is raised at step 200 and an alarm signal is raised to signal to an operator that there is a fault in operation of the device. The alarm signal is a combination of visual and sound signals that alerts an operator that there is a fault with the configuration of the device. If the measured pressure is found to be inside of those fault boundaries, then no adaptive alarm fault is raised at step 210. This represents the end of the algorithm. In either case, the algorithm ends at this point (220) and restarts at step 100.

Since the device is no longer in the start-up mode, at step 100 the method will move to step 110. At step 110, if it was determined in the previous method loop that there was an adaptive alarm fault, then the pressure measured during the previous method loop will not be used to update the long-term average of the pressure. Instead, the method will move straight to step 130 and determine again whether there is still a fault in operation of the device, or if the fault has been solved.

If It was determined in the previous method loop that there was not an adaptive alarm fault, then the pressure measured during the previous method loop will be used to update the long-term average of the pressure at step 120. The method will then move on to step 130, to update the upper and lower limits at steps 140 and 150 based on the updated long-term average of the pressure, and to measure and monitor the pressure again at steps 180-210.

Steps 100 to 220 are repeated as a continuous loop until interrupted by turning off of the device, changing of any attached equipment that requires a resetting of the method, changing of parameters implemented in the treatment, an initiation of a change in the treatment carried out by the device, or a user acknowledging an existing alarm fault signal. That is, in response to any of these events, the initialisation process will need to be repeated for 45 seconds once more.

FIG. 2 illustrates a method by which the fault boundaries for a gas parameter in a constant flow therapy device are dynamically updated during operation of the device according to a second embodiment, in order to determine whether there is an error in the operation of the device and then testing for a fault. It is worth noting that a long-term average of the sensor and an average deviation of the sensor are firstly determined by an initialisation process which is described in relation to FIG. 3 below.

Although illustrated differently, for simplicity of explanation, the method will be described here as beginning at step 340, since in reality the method is a continuous loop.

At step 340, an average reading is taken from a plurality of sensors measuring the same parameter. At step 350, it is calculated how far the average reading taken at step 340 deviates from the long-term average of the sensor, in order to determine a short-term deviation of the sensors. It is then determined whether the average reading taken at step 340 falls outside fault boundaries which are set as being three times the average deviation from the long-term average of the sensor. That is, it is determined whether the average reading taken at step 340 is greater than a first fault boundary or lower than a second fault boundary.

As with the method described in relation to FIG. 1, the method of this embodiment also employs the same compensating technique if it is determined that the fault boundaries resulting from three times the average deviation from the long-term average are too narrow. This shall not be repeated here for conciseness.

If the average reading taken at step 340 falls within the fault boundaries, then the method moves on to step 360, and the test is passed. That is, it is determined that there is no fault in operation. If the average reading taken at step 340 falls outside the fault boundaries, then the method moves on to step 370, and the test is failed. That is, it is determined that there is a fault in operation.

In either case, the method reverts to step 300 to perform another iteration of the method. At step 300, it is determined whether there is a currently a fault in the operation signalled by an alarm. If so, the method moves on to step 340, which allows updating of the long-term average of the sensor and the average deviation of the sensor to be skipped. This is implemented so that the long-term average of the sensor and the average deviation of the sensor, which are used for determining whether a fault occurs, are not skewed by the addition of a sensor value that itself has led to an error alarm signal.

Where there is no current alarm signal, the method moves on to step 310. At step 310 it is determined whether a second has elapsed since the long-term average of the sensor and the average deviation of the sensor were updated. This allows multiple sensor readings to be taken per second without using the processing power of updating the long-term average of the sensor and the average deviation of the sensor at every reading. Where one second has not elapsed, the method performs steps 340-370 again as previously described. It will be appreciated that in alternative embodiments, the long-term average of the sensor and the average deviation of the sensor may be updated each time a sensor reading is taken.

Where one second has elapsed since the long-term average of the sensor and the average deviation of the sensor were updated, the method proceeds to step 320 where the long-term average of the sensor is updated. Here, an average of the short-term average readings taken in the one second period since the long-term average was last updated is added to the long-term average of the sensor. The long-term average of the sensor comprises 32 readings which are made up of the previous 32 short-term averages, and the latest value replaces the oldest value of those 32 readings.

The method then proceeds to step 330, an average of the short-term deviations calculated in the one second period since the average deviation was last updated is added to the average deviation of the sensor. The average deviation of the sensor comprises 64 readings which are made up of the previous 64 averaged short-term deviations, and the latest value replaces the oldest value of those 64 readings.

Steps 300 to 370 are repeated as a continuous loop until interrupted by turning off of the device, changing of any attached equipment that requires a resetting of the method, changing of parameters implemented in the treatment, a change in the treatment carried out by the device is initiated, or user acknowledgement of an existing alarm fault signal. That is, in response to any of these events, the initialisation process illustrated in FIG. 3 will need to be repeated for 45 seconds once more.

At step 370, where it is determined that there is a fault in operation, an alarm signal is produced. The alarm signal is a combination of visual and sound signals that alerts an operator that there is a fault with the configuration of the device. FIG. 3 illustrates the initialisation process by which the long-term average of the sensor and the average deviation of the sensor are determined before the continuous method of FIG. 2 is implemented.

At step 400, an operator defines a second parameter, in this case the flow rate through the device, via a user interface associated with the device. At step 410, measurements of a first parameter, in this case the pressure at the device, are taken at 1 second intervals over a 45 second period by a pressure sensor positioned within the device. An average of the pressure readings taken is calculated and is set as a reference level for the pressure through the breathing system, the reference level being the long-term average of the sensor referred to in step 320.

Simultaneously, at step 420, the deviation of the pressure measurements taken over that 45-second period is determined relative to the average pressure calculated at step 410. The deviation of the measurements taken over that 45-second period may be an average of the deviation of each reading taken over that 45-second period (ie an average deviation of all the readings compared with the average reference level determined at step 410), or a maximum deviation from the reference level experienced over the duration of the 45-second period.

At step 430, the fault boundaries are defined as a function of the deviation of the pressure determined at step 420. Two fault boundaries are defined, a first fault boundary greater than the average pressure calculated in step 410 and a second fault boundary lower than the average pressure calculated in step 410. Worked examples of the calculations for defining the error parameter boundaries will be shown in more detail below.

In alternative embodiments, it is anticipated that alternative parameters may be set at step 400 and measured at steps 410, 420 and 340. In one example, the pressure may be set at step 400 and the flow rate may be measured at steps 410, 420 and 340.

In alternative embodiments, it is anticipated that the fault with the configuration of the device may be solved by the device itself, in which case the alarm signal may be an internal signal sent from a processor of the device to a controller of the device.

In alternative embodiments, only one fault boundary may be set at step 430. In this instance, the fault boundary may be greater or lower than the average pressure determined at step 410.

In alternative embodiments, in response to determining that there is a fault in operation of the device at step 370, operation of the device may cease until an operator has fixed the fault.

In alternative embodiments, the deviation recorded as a reference at step 330 may be a maximum deviation from the long-term average of the sensor.

Worked examples of steps 410 to 430 of FIG. 4 are presented in FIGS. 4, 5 and 6. For these examples, the values are given in arbitrary units for the sake of simplicity.

According to a first example, illustrated in FIG. 4, the pressure is measured at step 410 at 1 second intervals for 45 seconds, and the average pressure is determined to be 20. The pressure long-term average is therefore set as 20. The average deviation of the pressure measurements from the long-term average over that 45-second period is 2.4.

The fault boundaries in this example are set as an offset of three times the average deviation from the long-term average. The upper boundary is therefore set as 27.2, +7.2 from the long-term average, and the lower boundary is set as 12.8, −7.2 from the long-term average.

In the second and third examples, illustrated in FIGS. 5 and 6, rather than using the average deviation of the pressure measurements from the long-term average to calculate the upper and lower boundaries, a maximum deviation is used. Although not illustrated in FIG. 5 or 6, this may require the upper and lower boundaries to be offset by a lesser multiple of the calculated deviations.

In FIG. 5, the pressure is measured at step 410 for 45 seconds, and the average pressure is determined to be 20. The long-term average of the sensor is therefore set as 20. The pressure measurements fluctuate over that 45-second period between values of 18 and 23. The maximum deviation of the pressure measurements from the long-term average is therefore recorded as 3.

The fault boundaries in this example are set as an offset of three times the maximum deviation from the long-term average. The upper boundary is therefore set as 29, +9 from the long-term average, and the lower boundary is set as 11, −9 from the long-term average.

In FIG. 6, the pressure is measured at step 410 for 45 seconds, and the average pressure is determined to be 20. The pressure long-term average is therefore set as 20. The pressure measurements fluctuate over that 45-second period between values of 18 and 23. In this example, the maximum deviation of the pressure measurements from the long-term average is recorded as 2 in the negative direction and 3 in the positive direction.

The fault boundaries in this example are set as an offset of three times the maximum deviation in that specific direction from the long-term average. The upper boundary is therefore set as 29, +9 (3×3) from the long-term average, and the lower boundary is set as 14, ×6 (3×−2) from the long-term average.

1-42. (canceled)

43. A respiratory apparatus for supplying gas to a breathing system, the respiratory apparatus comprising:
at least one sensor configured to take a series of measurements of a first parameter of the breathing system and configured, in at least one update procedure, to take one or more further measurements of the first parameter;
a controller configured to set a fault boundary for the first parameter, the fault boundary being dependent on a plurality of the measurements of the first parameter, and configured, in the at least one update procedure, to update the fault boundary, the updated fault boundary dependent on an updated set of measurements of the first parameter, the updated set of measurements of the first parameter including at least one of the further measurements of the first parameter.

44. The respiratory apparatus according to claim 43, wherein the controller is configured to set the fault boundary for the first parameter to be offset from a measurement parameter that is dependent on a plurality of the measurements of the first parameter.

45. The respiratory apparatus according to claim 44, wherein the offset of the fault boundary from the measurement parameter is a predetermined amount or an amount that is dependent on a variance factor of a plurality of the measurements.

46. The respiratory apparatus according to claim 44, wherein after taking one or more further measurements of the first parameter, the controller is configured to update the measurement parameter, the updated measurement parameter being dependent on at least the one or more further measurements of the first parameter.

47. The respiratory apparatus according to claim 46, wherein the controller is configured to set the updated fault boundary to be offset from the updated measurement parameter.

48. The method according to claim 47, wherein the offset of the fault boundary from the measurement parameter is an amount that is dependent on a variance factor of a plurality of the measurements, and after the one or more further measurements of the first parameter are taken, the controller is configured to update the variance factor, the updated variance factor being dependent on the updated set of measurements of the first parameter, and the offset of the updated fault boundary from the updated measurement parameter is an amount that is dependent on the updated variance factor.

49. The respiratory apparatus according to claim 48, wherein the updated variance factor is dependent on at least one of the further measurements of the first parameter.

50. The respiratory apparatus according to claim 44, wherein the measurement parameter is an average of a plurality of the measurements and/or wherein the updated measurement parameter is an average of a plurality of the updated set of measurements.

51. The respiratory apparatus according to claim 43, wherein the updated set of measurements comprises at least one of the measurements upon which the fault boundary being updated is dependent and at least one of the further measurements, and wherein the at least one of the further measurements replaces an equivalent number of the earliest measurements upon which the fault boundary being updated is dependent.

52. The respiratory apparatus according to claim 43, wherein the controller is further configured to compare a measurement of the first parameter with either the fault boundary, or once the fault boundary has been updated, the updated fault boundary, and determine a fault with the operation of the breathing system in response to the measurement being outside of the fault boundary or the updated fault boundary.

53. The respiratory apparatus according to claim 52, wherein in response to determining a fault with the operation of the breathing system, the controller is configured to indicate the fault to a user.

54. The respiratory apparatus according to claim 43, wherein the sensor and/or the controller are configured to repeat the update procedure at least once, wherein the fault boundary being updated in each subsequent update procedure is the updated fault boundary from the previous update procedure.

55. The respiratory apparatus according to claim 54, wherein the sensor and/or the controller are configured to repeat the update procedure at intervals during operation of the breathing system, including at least a period during which gas is supplied to the breathing system, and/or the period during which treatment is being provided to a patient, wherein at least one of the intervals is less than 10 seconds.

56. A respiratory apparatus according to claim 43, wherein the at least one sensor is configured to take the series of measurements of the first parameter of the breathing system during operation of the breathing system, and/or the controller is configured to set the fault boundary for the first parameter during operation of the breathing system.

57. A respiratory apparatus according to claim 43, wherein the respiratory apparatus is configured to commence supplying gas to the breathing system before the at least one sensor takes the series of measurements of the first parameter of the breathing system.

58. A respiratory system comprising the respiratory apparatus of claim 43 and a breathing system in fluid communication with the respiratory apparatus, the breathing system comprising a patient interface arranged to supply gas to a patient in use, and a breathing tube arranged to deliver the gas supply from the respiratory apparatus to the patient interface.

59. A method of detecting a fault in a breathing system, the method comprising the steps of:
(a) taking a series of measurements of a first parameter of the breathing system; and
(b) setting a fault boundary for the first parameter, the fault boundary being dependent on a plurality of the measurements of the first parameter; wherein the method includes at least one update procedure comprising the steps of:
(c) taking one or more further measurements of the first parameter; and
(d) updating the fault boundary, the updated fault boundary being dependent on an updated set of measurements of the first parameter, the updated set of measurements of the first parameter including at least one of the further measurements of the first parameter.

60. A method of detecting a fault in a breathing system, the method comprising the steps of:
(a) supplying a gas to the breathing system;
(b) taking a series of measurements of a first parameter of the breathing system;
(c) setting a fault boundary for the first parameter, the fault boundary being dependent on a plurality of the measurements of the first parameter, wherein a second parameter of the breathing system is controlled during supply of the gas to the breathing system, and the first parameter of the breathing system is dependent on the second parameter and the breathing system, such that a change in the first parameter may be indicative of a fault in the breathing system.

61. A method as claimed in claim 60, wherein the second parameter is flow rate or pressure within the device or the breathing system.

62. A method as claimed in claim 60, wherein the first parameter is any of flow rate, back pressure within the breathing system, or patient pressure.
